(19) United States
(12) Patent Application Publication
(10) Pub. No.: US 2008/0006782 A1
(43) Pub. Date: Jan. 10, 2008

(54) Device For Uv-Irradiating Of Human's Cutaneous Covering Vertical Solarium

(75) Inventors: Sergey Evgenievich Mikhailov, Moscow (RU); Valentin Afanasievich Baikulov, Moscow (RU); Dmitry Yurievich Chinikhin, Moscow Region (RU)

Correspondence Address: WILLIAM COLLARD;COLLARD & ROE, P.C., 1077 NORTHERN BOULEVARD, ROSLYN, NY, 11576, US

(73) Assignee: Sergey Evgenievich Mikhailov, Moscow (RU)

(21) Appl. No.: 10/590,373

(22) PCT Filed: Feb. 4, 2005
(86) PCT No.: PCT/RU05/00038

§ 371 Date: Dec. 27, 2006

(30) Foreign Application Priority Data
Feb. 24, 2004 (RU) 2004105108

(51) Int. Cl. A61N 5/06

(52) U.S. Cl. 250/494.1

(57) ABSTRACT

This invention relates to medical engineering, more particularly, to therapeutically applicable light-irradiating devices used for treatment of skin diseases, as well as for generating vitamin D₃ and preventing various forms of osteoporosis. In addition, the device can find application at beauty shops and sunless-tanning studios for cosmetic purposes.

A technical problem to be solved by the present invention resides in increasing the efficiency of the device.

The problem is solved due to reducing the amount of the lamps to ‘n’ and substituting the plain circular cylinder-shaped reflector by a reflector comprised of 2n alternating areas of involute cylinder-shaped surfaces of two types integrated into a single surface, the evolutes of said surfaces are closed curves which limit the convex transverse sections of the lamp and absorber in contemplation, respectively, which reflector is interposed between the lamps and the solarium body.

Hence carrying the invention into effect enables one to reduce electric power consumption of the vertical solarium three- to fourfold, the value of the UV-radiation flux remaining unaffected.

The present invention relates to medical engineering, more particularly, to therapeutically applicable light-irradiating devices used for treating skin diseases, such as psoriasis, Kaposi's disease, vitiligo, and others), as well as for generating vitamin D₃ and preventing various forms of osteoporosis. In addition, the device can find utility when used at beauty shops and sunless-tanning studios for cosmetic purposes.

One prior-art device for UV-irradiation of human's cutaneous covering, wherein the user assumes the standing posture, operating in the UV-radiation spectrum A (315-400 nm) (hereinafter referred to as “UF-A spectrum”)—vertical solarium available SunVision by the firm ALISUN (both from the Netherlands) (cf. the supplement below, by the manufacturer's prospectus entitled “New vertical solarias Sun-Vision”, said device comprising a body having a door and accommodating 48 fluorescent lamps for taking sunless tan, each being 2 m long and having a power output of 180 W. The lamps are spaced apart at an equal angular pitch round a common axis which is at the same time the solarium's axis, arranged parallel thereto and equidistantly therewith; in addition, the lamps have a common mirror reflector which is interposed between the lamps and the body and is spaced 10 mm apart from the lamp surface; the reflector appears as a circular cylinder having an inner mirrored surface. The device is further provided with an air-cooling system for the lamps and user. However, the device under discussion suffers from too a low efficiency and high power input.

### Estimation Of The Prototype Efficiency

Taking into consideration an axial symmetry of the vertical solarium in question, user's ability to assume various positions during the session, and bearing in mind that the breadth of human's shoulder and pelvis in a majority of cases approximates 500 mm, it would be true and correct to imagine the user as a conventional convex radiation absorber appearing as a circular cylinder 500 mm in diameter and arranged coaxially with the solarium.

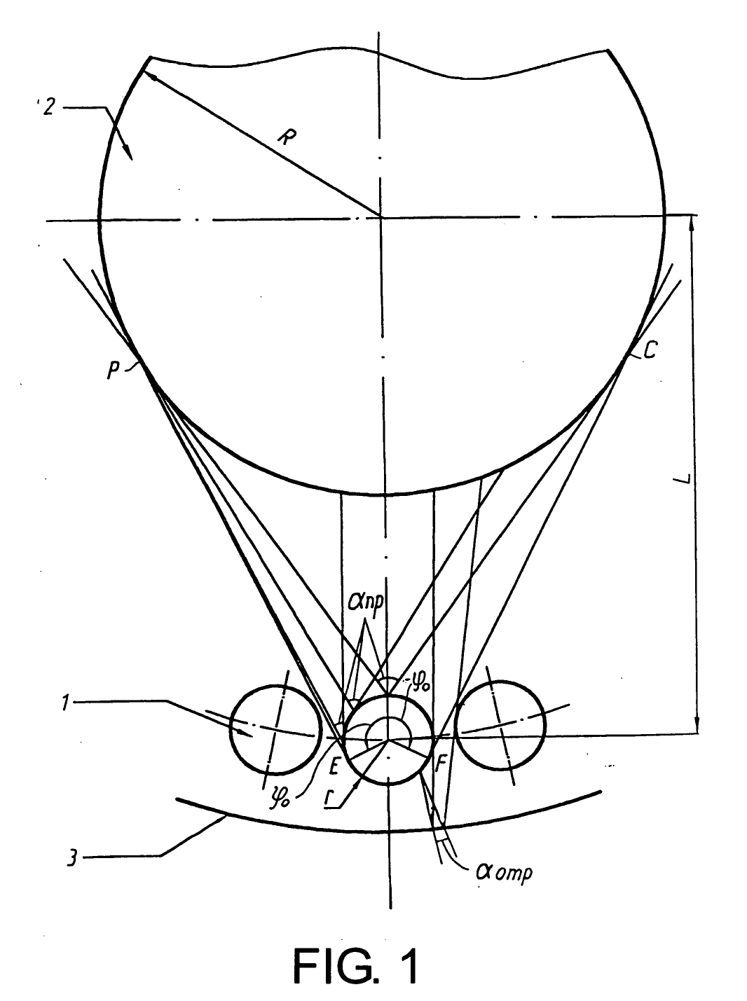

The simplifying assumptions thus made result in an axisymmetrical design model of a vertical solarium for the studying of which it is sufficient to consider a bidimensional problem (FIG. 1).

Let us assume that portion of the UV radiation emitted by a lamp 1 which is incident upon an absorber 2 either directly from said lamp or after having been reflected to be a useful one, and its share in a total radiation is assumed to be an optical efficiency of the lamp (hereinafter referred to as “efficiency”) which is numerically equal to the efficiency of the whole solarium on account of symmetry of the model (loss for ventilation, decorative boost lighting, and so on are left out of the given evaluation). In terms of illumination power the solarium efficiency is described by the formula:

(1) Efficiency=100%(Φ_{UVdir}+Φ_{UV}_{ref})/φ_{UV}, wherein:

Φ_{UVdir}—direct (non-reflected) UV radiation flow incident upon the absorber;

$${Φ}_{UVdir} = {\int }_{{λ}_{1}}^{{λ}_{2}} {Φ}_{UVdir} d λ ;$$

ΦUV_{ref}—reflected UV radiation flow incident upon the absorber;

$${Φ}_{UVref} = {\int }_{{λ}_{1}}^{{λ}_{2}} {Φ}_{UVref} d λ ;$$

Φ_{UV}—UV radiation flow emitted by the lamp (ratio between the energy transferred by the radiation and the transfer time exceeding considerably the oscillation period, W):

$${Φ}_{UV} = {\int }_{{λ}_{1}}^{{λ}_{2}} {Φ}_{UV} d λ ;$$

wherein:

Φλ_{UV}—spectral flow density (radiation flow per unit wavelength interval, W/nm).

Forasmuch as the lamp radiates light in every direction (i.e., diffusely) within the whole wavelength range of interest to us, equation (1) may be written in terms of angular values:

$$Efficiency = \frac{{α}_{dir . av .} + {α}_{ref . av} {K}_{ref .}}{{2}_{Π}} 100 \% \qquad ( 2 )$$

wherein:

α_{dir.av}—average magnitude of angle α_{pr.};

α_{dir.}—angle at which absorber is seen from the point on the surface of the luminous element of the lamp;

α_{ref.av}—average magnitude of angle α_{ref.};

α_{ref.}—angle at which an airgap between lamps im reflector 3 is seen from the point on the surface of the incandescent body of the lamp;

K_{ref.}—total reflection coefficient of the reflector.

It is a phosphor layer that serves as the luminous element in fluorescent lamps made use of in solaria, said layer following the geometric shape of the gas-discharge tube, and angles α_{dir.av} and α_{ref.av} are found from the following relationships:

$$\begin{matrix}{α}_{dir . av} = \frac{{\int }_{- {ϕ}_{0}}^{{ϕ}_{0}} α ( ϕ ) d ϕ}{2 {ϕ}_{0}} & ( 3 ) \\ {α}_{dir . av} = \frac{2 {\int }_{{ϕ}_{1}}^{{ϕ}_{2}} α ( ϕ ) d ϕ}{ {ϕ}_{2} - {ϕ}_{1}} & ( 4 )\end{matrix}$$

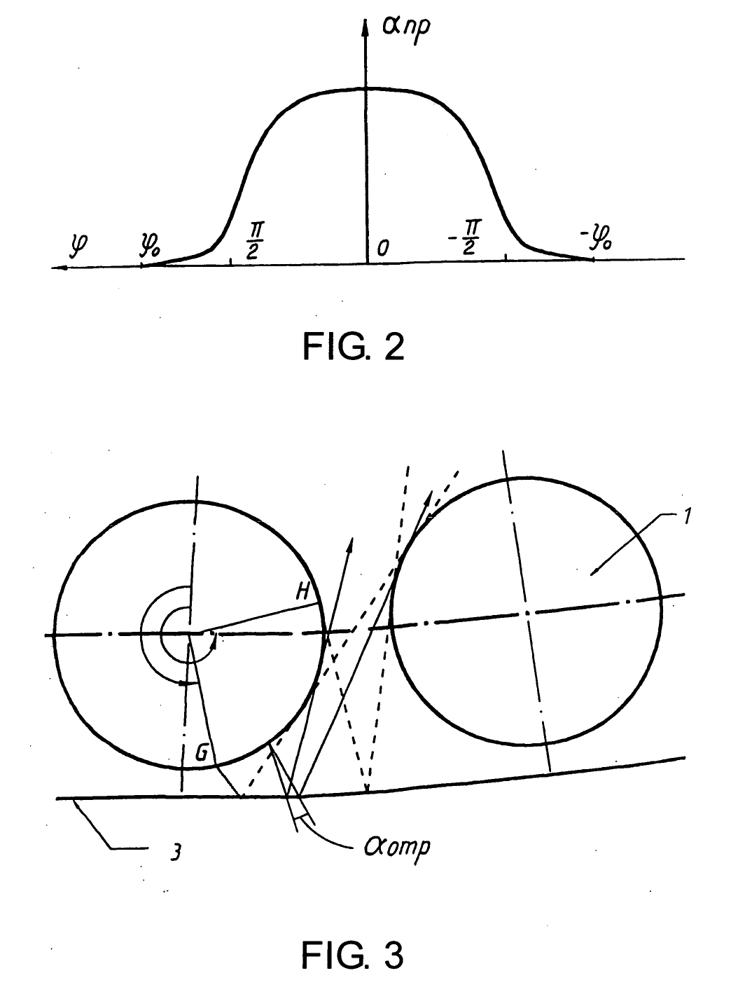

The limits of integration in formula (3) φₒ and _φₒ are angular coordinates of “points of sunset” E and F, i.e., such points on the lamp surface that lie on common tangents EP and CF of the lamp and absorber, respectively. All points on the lamp surface having coordinate φ are larger than φₒ but less than 2π-φₒ do not irradiate the absorber directly. The nature of dependence of angle α_{dir}. on the angular coordinate of the radiating point on the surface of the lamp luminous element are illustrated in FIG. 2.

φₒ=π/2+Arcsin((R−r)/L) (5)

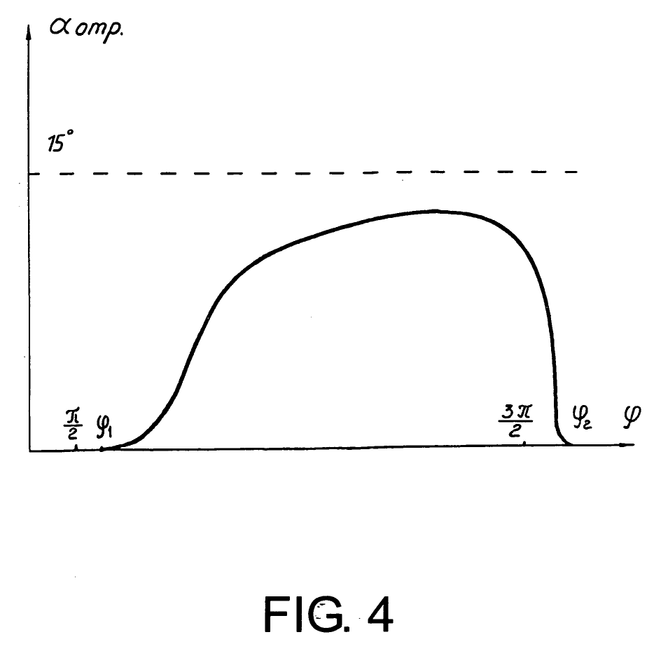

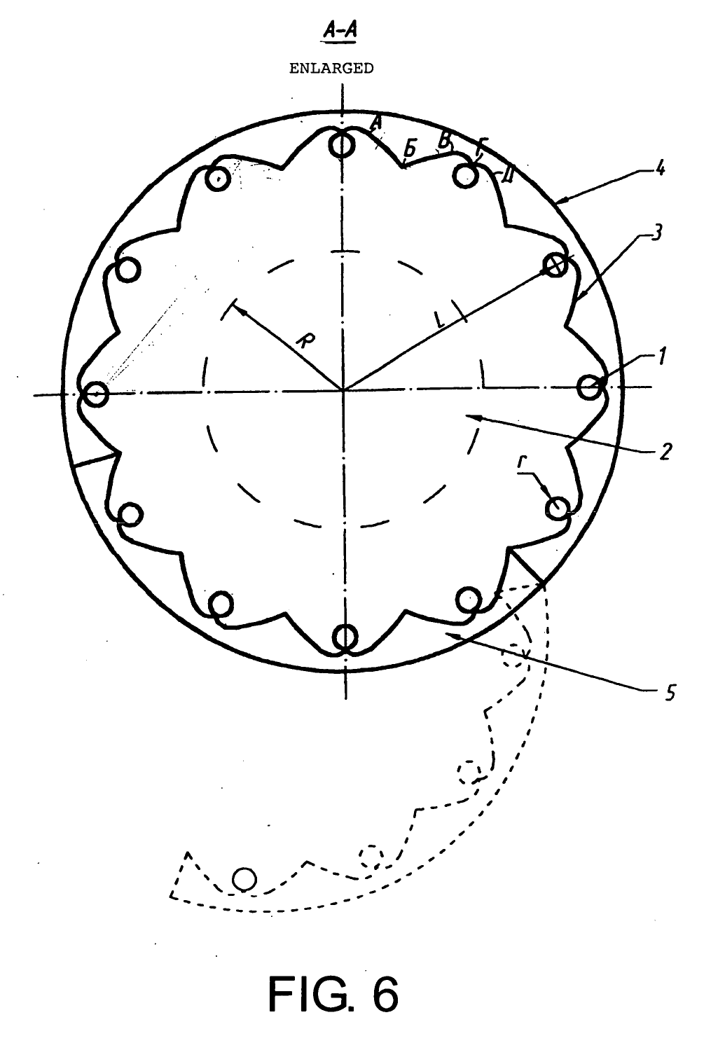

Angular coordinates of the “points of sunset” G and H for calculating α_{ref.av} are also determined on the basis of the solarium geometry whereby they are therein omitted. A diagram of pathways of the light rays between the lamps after their having been reflected from the reflector is shown in FIG. 3. The nature of dependence of angle α_{ref}. On the position of a point on the lamp surface is presented in FIG. 4.

Having applied formulas (2), (3) and (4) for estimating efficiency of the prototype having the following dimensions: L=440 mm, R=250 mm, r=20 mm, we shall obtain:

α_{dir.av}=70.52° (1.23 rad)

α_{ref.av}=8.95°×2=17.890 (0.31 rad)

K_{ref}=1 (assuming the mirror to be ideal) Efficiency=24.59%.

Herein Φ_{UVref}=0.0497 Φ_{UV} (0.0447 Φ_{UV} for a mirror from pure aluminum having K_{ref}=0.9. However, reflector contribution to the efficiency of the device is but rather small, since any ray that has failed to get incident upon the absorber after first reflection will yet not be absorbed by the absorber, because the lamp-to-lamp airgaps are small and the light ray has no opportunity to have reflection twice. It is easy to verify the fact by making rather simple geometric constructions.

The results of estimations performed on the basis of a bidimensional model are corroborated by the results of measurement of irradiance in UV-A spectral range carried out by the authors on the prototype.

Hence we have ascertained that efficiency of the prototype is as low as 25% and that the reflector made use of therein directs as low as 5% of the total lamp radiation onto the absorber.

Thus, the present invention is aimed at solving a technical problem which resides in attaining higher efficiency of the device.

The technical problem of the invention is solved by reducing the amount of the lamps used and substituting the circular cylinder-shaped reflector made use of in the prototype, by a reflector comprised of 2n alternating areas of involute cylinder-shaped surfaces of two types integrated into a single surface, appearing from the evolutes of which are closed curves which limit the convex transverse sections of the absorber and lamp in contemplation.

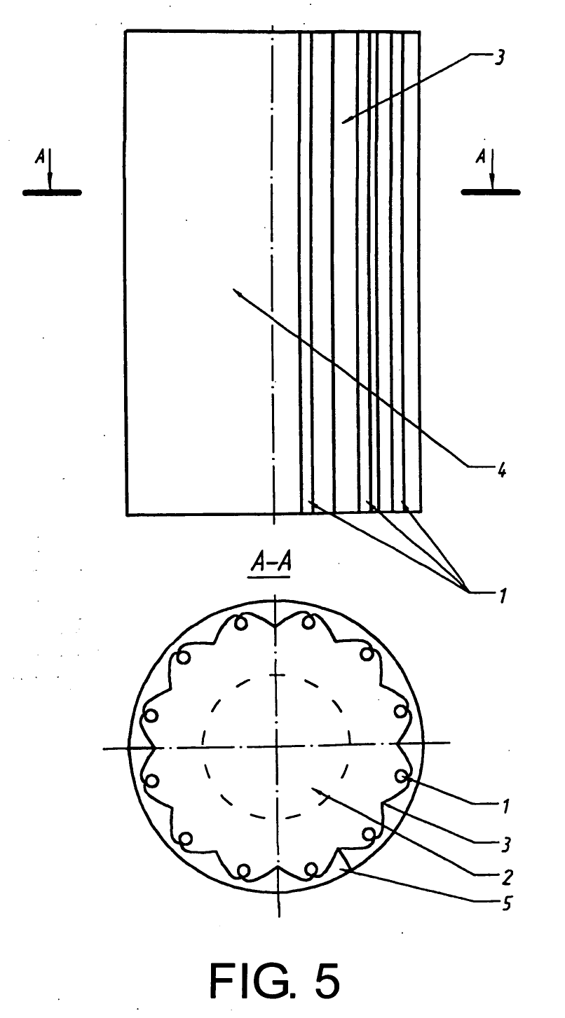

The essence of the invention is illustrated by a schematic diagram of the device with 12 lamps (FIG. 5) and a luminous flux equal to that in the 48-lamp prototype. A cross-section shown in FIG. 6 is conventionally enlarged.

Principal structural components of the solarium are as follows: a vertically oriented body 4 having a door 5, n fluorescent lamps for taking sunless tan, said lamps being spaced apart at an equal angular pitch round a common axis which is at the same time the solarium's axis, arranged parallel thereto and equidistantly therewith. A mirror reflector 3 is interposed between the body 4 and the lamps 1, said reflector appearing as a cylinder coaxial with the body and comprised of 2n alternating areas of involute cylinder-shaped surfaces of two types.

The areas of the first-type surface are disposed immediately behind the lamps 1 (curve BΓ [symbol] , FIG. 6) and appear as a portion of an involute cylinder-shaped surface generated by moving a straight line parallel to the solarium body axis lengthwise the straight line segments of the unlike branches of the involute of a closed curve which limits the convex transverse section of the lamp.

The areas of the second-type surface are interposed between the lamps (curve ABB in FIG. 6) and appear as a portion of an involute cylinder-shaped surface generated by moving a straight line parallel to the solarium axis lengthwise the segments of the unlike branches of a convex closed curve which limits the transverse section of the conventional absorber.

The areas of the first-type and second-type surfaces are mated gently at the point B. It is due to the fact that the normal to an involute is thereto a tangent to the evolute by definition, that the herein-proposed reflector shape provides for both complete radiation emergence by preventing it from being reflected back onto the lamp, and total reflection to the absorber of all rays which have failed to get incident thereupon directly from the lamp.

The herein-proposed solarium may have as high efficiency as about 100%. It is noteworthy that efficiency of the solarium depends on the number of lamps used, since some of the luminous rays are absorbed after having got incident upon other lamps. Thus, for an ideal reflector the solarium having 6 lamps features an efficiency of 98.4%, the solarium with 12 lamps, an efficiency of 89.2%, and the solarium with 16 lamps, an efficiency of 79.7%.

Consequently, in order to get absorber illuminance in the UF-A spectrum equal to that of the prototype, it suffices 12 lamp of the same power, i.e., carrying the present invention into effect enables one to reduce electric power consumption approximately 3.6 times compared with the prototype.

1. A device for UV-irradiating of human's cutaneous covering, i.e., a vertical solarium comprising a cylinder-shaped body closed along the perimeter thereof provided with a door and accommodating ‘n’ fluorescent lamps for taking sunless tan, said lamps being spaced apart at an equal angular pitch round an axis which is at the same time the solarium's axis, and being arranged parallel thereto and equidistantly therewith, said device further comprising a cylinder-shaped mirror reflector which is coaxial with the solarium body and is interposed between the lamps and said body, CHARACTERIZED in that the reflector is comprised of 2n alternating areas (integrated into a cylinder) of first-type and second-type involute cylinder-shaped surfaces the evolutes of which are closed curves which limit the convex transverse sections of the lamp and conventional absorber, respectively, each area of the first-type surface is disposed immediately behind each lamp and appears as a portion of an involute cylinder-shaped surface generated by moving a straight line parallel to the solarium body axis lengthwise the involute of a closed curve which limits the convex transverse section of the lamp, and each area of the second-type surface is disposed between the lamps and is a portion of an involute cylinder-shaped surface generated by moving a straight line parallel to the solarium body axis lengthwise the involute of a closed curve which limits the transverse section of the conventional absorber.
